(19) United States
(12) Patent Application Publication
(10) Pub. No.: US 2017/0333159 A1
(43) Pub. Date: Nov. 23, 2017

(54) DENTAL APPLIANCE

(71) Applicant: MEDRIAN OY, Joensuu (FI)

(72) Inventors: Riitta Pietarinen, Joensuu (FI); Anne Helin, Joensuu (FI)

(21) Appl. No.: 15/526,501

(22) PCT Filed: Nov. 20, 2015
(86) PCT No.: PCT/FI2015/050809

§ 371 Date: May 12, 2017

(30) Foreign Application Priority Data
Nov. 21, 2014 (FI) 20146023

(51) Int. Cl. A61C 5/90; A61C 17/06; A61B 13/00

(52) U.S. Cl. CPC A61C 5/90; A61B 13/00; A61C 17/043

(57) ABSTRACT

A dental appliance comprising: a tongue support element (1) with a trough (2) for the tongue, the trough having a concave inner surface and a convex outer surface; a tooth support element (3) with an upper and a lower bite surface (4a, 4b), which are arranged on the outer surface side of the tongue trough (2) so that the upper bite surface (4a) coincides with the lower bite surface (4b), only on an opposite side of the tongue trough (2), and so that a first end (5) of the inner surface of the tongue trough is between the upper and the lower bite surfaces (4a, 4b) of the tooth support element (3). The inner surface (6) of the tongue trough is directed sideways in relation to the tooth support element (3), the inner surface (6) of the tongue trough being arranged to receive a patient's tongue and to position it to a side of the oral cavity, partly between the bite surfaces (4a, 4b).

### BACKGROUND

The invention relates to a tooth and tongue support.

Keeping the mouth open is a common problem in dental care because the patient's muscles get tired during a long treatment session. Another problem may be caused by the patient's tongue, which may at least occasionally obstruct the area under treatment.

Prior art knows different accessories for keeping the mouth open and/or the tongue away from the area under treatment. However, a fully satisfying solution to the above problems has not been presented.

### BRIEF DISCLOSURE

A tooth and tongue support of the invention is characterized by what is stated in the independent claim. The other embodiments of the invention are characterized by what is disclosed in the rest of the claims.

Inventive embodiments are also disclosed in the specification and the drawings of this application. The inventive content of the application may also be defined differently than in the claims presented below. The inventive content may also consist of a plural number of separate inventions, particularly if the invention is examined in the light of disclosed or implicit subtasks or from the point of view of gained advantages or groups of advantages. In that case some of the definitions contained in the claims below may be irrelevant to the separate inventive ideas. The features of the different embodiments of the invention may be applied in connection with other embodiments within the scope of the basic inventive idea.

The tooth and tongue support now under discussion comprises a tongue support element with a trough for the tongue, the trough having a concave inner surface and a convex outer surface; a tooth support element with an upper and a lower bite surface, which are arranged on an outer surface side of the tongue trough so that the upper bite surface coincides with the lower bite surface, only on an opposite side of the tongue trough, and so that a first end of the inner surface of the tongue trough is between the upper and the lower bite surfaces of the tooth support element, and the inner surface of the tongue trough is directed sideways in relation to the tooth support element, the inner surface of the tongue trough being thus arranged to receive a patient's tongue and to position it to a side of the oral cavity, partly between the bite surfaces.

An advantage of this is that the area under treatment is made free of obstruction in a manner that is pleasant to the patient.

In the following, features of some embodiments of the invention are discussed in a random order.

According to an embodiment, a bite surface has a basic shape that is curved in a longitudinal direction thereof. An advantage of this is that the position of the tooth and tongue support in the mouth may be adjusted by turning the support in the longitudinal direction of the bite surface.

According to an embodiment, a bite surface comprises protrusions that may be arranged in a transverse direction, for example. An advantage of this is that the support stays better in place against an uneven line of teeth.

According to an embodiment, a bite surface comprises a raised edge portion. An advantage of this is that the risk of the bite surface from slipping away from contact with the teeth can be reduced.

According to an embodiment, the tongue trough has a lower portion that is arranged to extend past the lower bite surface. An advantage of this is that the tongue trough efficiently prevents the tongue from entering the area under treatment and that the tongue is protected against possible accidents, such as the slipping of the drill or an instrument.

According to an embodiment, the tooth and tongue support comprises a suction channel connector arranged to the tongue support element to receive a suction tube. An advantage of this is that the tooth and tongue support also serves as a holding fixture for the suction tube and as an element keeping it in place, and also that the suction tube is prevented from accidentally slipping into the pharynx, whereby the personnel is better able to concentrate on other tasks required by the treatment.

According to an embodiment, the tooth and tongue support comprises a suction trough arranged into the tongue trough and forming a recess that extends to the outer surface side of the tongue trough, to which recess said suction channel connector is connected and thus fluids collecting into the recess may be evacuated through the suction tube. An advantage gained by this is more efficient removal of fluids.

According to an embodiment, the inner surface of the suction trough comprises at least one groove connecting with said recess. An advantage of this is that even minor amounts of fluids can be made to flow into the suction area of the suction tube.

According to an embodiment, the tooth and tongue support is a single uniform piece of material. An advantage of this is that the support is easy and quick to handle.

### LIST OF FIGURES

The matter will be discussed in closer detail in the accompanying drawings, in which

FIG. 1 is a schematic side view showing a tooth and tongue support from an inner surface side of a tongue trough,

FIG. 2 is a schematic front view of the tooth and tongue support of FIG. 1,

FIG. 3 is a schematic view from above of the tooth and tongue support of FIG. 1, and

FIG. 4 is a schematic diagonal rear perspective view from below of the tooth and tongue support of FIG. 1.

For the sake of clarity, the figures show the tooth and tongue support in a simplified manner. In the figures, like reference numerals identify like elements.

### DETAILED DESCRIPTION

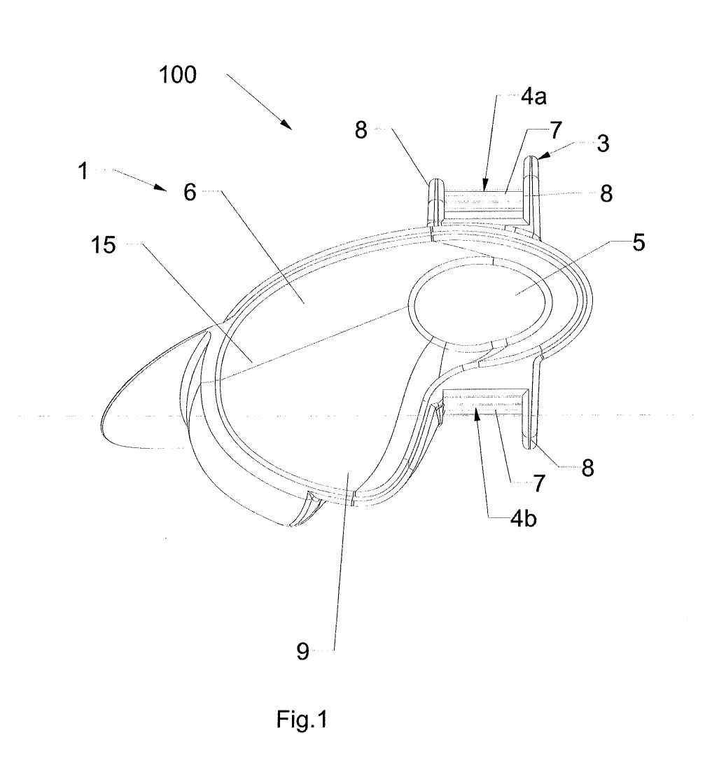

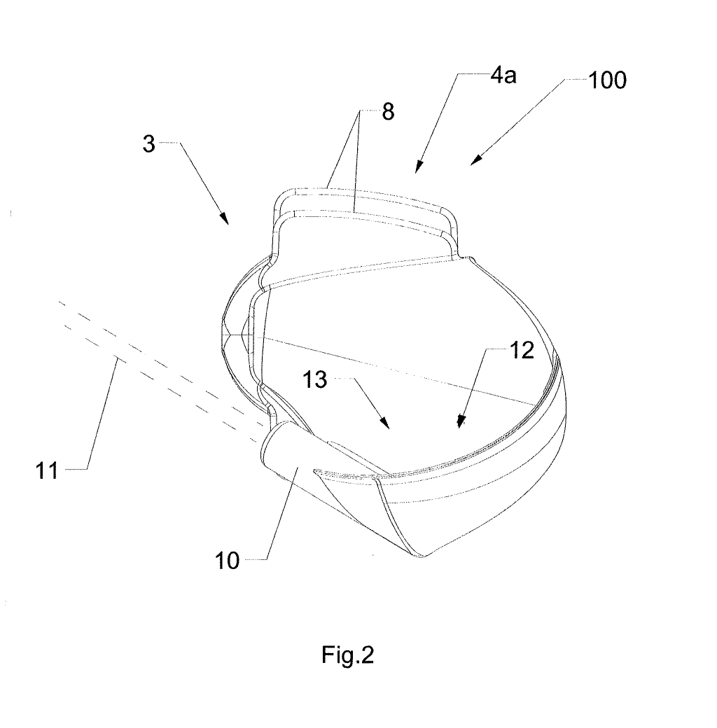

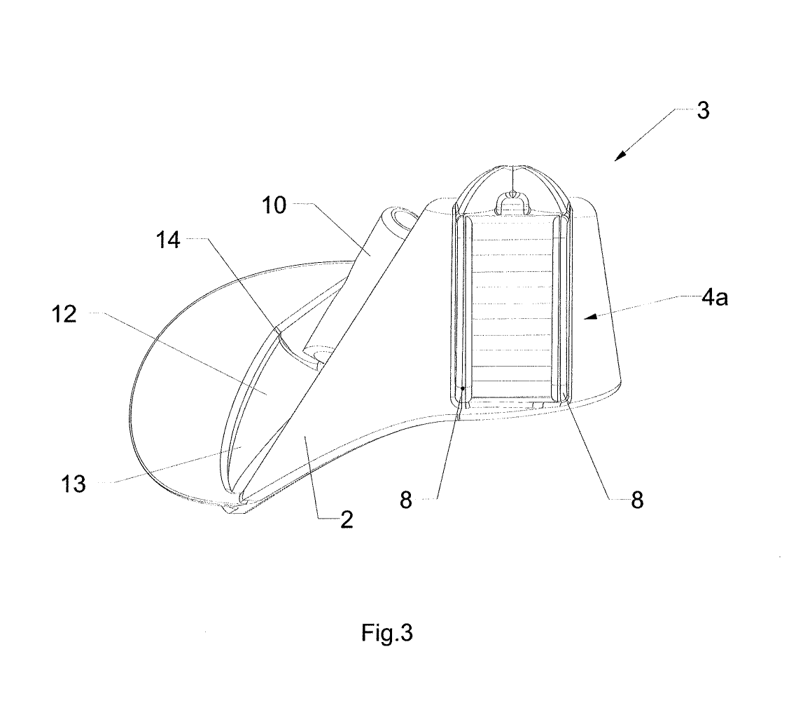

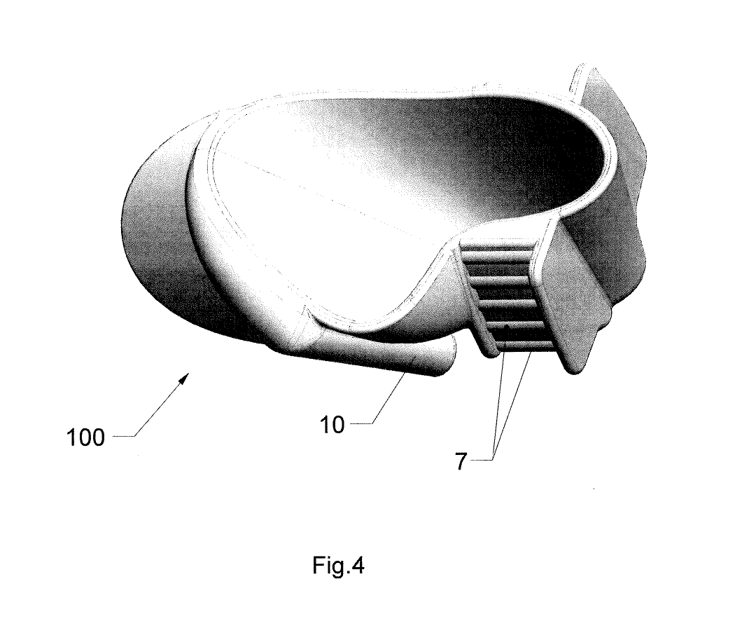

FIG. 1 shows schematically a tooth and tongue support from an inner surface side of a tongue trough, FIG. 2 from the front, FIG. 3 from above, and FIG. 4 as a diagonal rear perspective view from below.

The tooth and tongue support 100 is preferably made of one and the same piece of material, which is preferably made of a resilient material, such as plastic or elastomer or a mixture containing these. The manufacturing material may comprise e.g. thermoset or thermoplastic, such as polyolefin, e.g. polypropylene, and/or material marketed under trade name Dryflex.

The tooth and tongue support 100 may be disposable or reusable after disinfection. According to another idea, the tooth and tongue support 100 may be made of two or more parts to be connected together, the manufacturing materials of which may be the same or differ from another.

Because of its resilience, the tooth and tongue support 100 may be arranged according to personal features of the mouth to an optimal position in the oral cavity. Moreover, the resilient material is pleasant to the patient.

The tooth and tongue support 100 comprises a tongue support element 1 with a trough 2 for the tongue, the trough having a concave inner surface and a convex outer surface, and a tooth support element 3 with an upper and a lower bite surface 4a, 4b.

Said bite surfaces 4a, 4b are arranged to the convex outer surface of the tongue trough 2 so that the upper bite surface 4a coincides with the lower bite surface 4b, only on an opposite side of the tongue trough 2.

The inner surface 6 of the tongue trough is directed sideways in relation to the tooth support element 3. A first end 5 of the inner surface 5 of the tongue trough is between the upper and the lower bite surfaces 4a, 4b of the tooth support element 3, which feature further enhances resilience between the bite surfaces 4a, 4b. A second end 15 of the inner surface 6 of the tongue trough extends to a distance from the first end 5 that allows the patient's tongue to be substantially entirely covered by the tongue trough 2.

The concave inner surface 6 of the tongue trough receives the patient's tongue and positions it to one side of the oral cavity, partly between the bite surfaces 4a, 4b.

As seen in FIG. 1, a lower portion 9 of the tongue trough 2 extends past level L of the bite surface 4b, i.e. below it. It is thus possible to preferably arrange the lower portion 9 against the floor of the oral cavity and/or against the gums of the lower teeth so as to make sure that the tongue does not escape from the space reserved for it by passing under the tongue trough 2.

The dimensioning of the tooth and tongue support 100 and the details of its design have been advantageously selected in accordance with the specific features of the oral cavity to make it suitable for the oral cavity of most patients. However, due to natural variation in the shapes and sizes of oral cavities, a series of tooth and tongue supports 100 of different sizes and shapes may be provided. According to another idea, the tooth and tongue support 100 is tailor-made according to measurements taken from the patient's oral cavity.

The tooth and tongue support 100 shown in the figure is asymmetric so that it may be used on the right side of the oral cavity. In that case the tooth and tongue support 100 positions the tongue to the left side of the oral cavity, partly between the left upper and lower teeth as a result of which ample space for treatment is left on the right side and the front part of the oral cavity. Due to the asymmetry, a separate tooth and tongue support 100 may be made for treatment to be carried out on the left side of the oral cavity, which support may be a mirror image of the tooth and tongue support 100 shown in the figure.

The bite surface 4a, 4b may have a basic shape that is convexly curved in its longitudinal direction. A curved basic shape may help to arrange the support 100 to an optimal position in the oral cavity. However, said curvature is not indispensable: the bite surface 4a, 4b may be at least substantially straight, for example.

In the figures the bite surface 4a, 4b comprises protrusions 7 arranged in a transverse direction. With the protrusions 7 the support 100 may stay in place better. There are naturally also other ways of arranging the protrusions 7, for example in a longitudinal direction of the bite surface 4a, 4b, or they may be left out altogether.

The bite surface 4a, 4b shown in the figures comprises a raised edge portion 8 on both sides thereof. This reduces, among other things, the possibility of the bite surface 4a, 4b from slipping away from between the teeth.

The tooth and tongue support 100 shown in the figures has a suction channel connector 10 arranged to the tongue support element 1, to which connector a suction tube 11 may be fastened. It should be noted at this point that the tooth and tongue support 100 may also be implemented without the suction channel connector 10.

During treatment, such as filling of a tooth, carried out in the oral cavity, fluids, such as saliva and/or water, collect into the oral cavity. The fluids must be evacuated from time to time so that the patient would not feel uncomfortable or to keep dry the area under treatment. It is known in the art to use a suction tube 11 for this purpose.

The tooth and tongue support 100 shown in the figures further comprises a suction trough 12 arranged into the tongue trough and forming a recess 13 that sets on the outer surface side of the tongue trough 2. The suction trough 12 preferably has a resilient structure so that it may be positioned in an optimal manner to the oral cavity according to the patient's individual features.

Fluids collecting into the oral cavity collect into the suction trough 12 and its recess 13. The suction channel connector 10 is arranged to be in connection with said recess 13 so that fluids collecting into the recess 13 may be evacuated through the suction tube 11. According to an idea, the suction tube 11 may be arranged to the recess 13 by means of the suction channel connector 10. According to another idea, the suction channel connector 10 reaches the recess 13, in which case the suction tube 11 does not necessarily need to be extended all the way to the recess 13.

According to an idea, the inner surface of the suction trough 12 comprises one or more grooves 14 connecting with said recess 13. The groove 14 enhances the flow of fluids to be removed into the recess 13 especially when fluids collect slowly in the suction trough 12. With the arrangement described here it is possible to direct the suction to the lowermost point of the suction trough 12, i.e. to the point where the fluids naturally flow.

However, it should be noted that the tooth and tongue support 100 may also be implemented without the suction trough 11.

In some cases, features disclosed in this application may be used as such, regardless of other features. On the other hand, when necessary, features disclosed in this application may be combined in order to provide different combinations.

The drawings and related disclosure are only intended to illustrate the inventive idea. It will be apparent to a person skilled in the art that the invention is not restricted to the above-described embodiments disclosing the invention through some examples, but various modifications and different applications of the invention are feasible within the inventive idea defined in the accompanying claims.

### REFERENCE MARKINGS

1 tongue support element
2 tongue trough
3 tooth support element
4a, 4b bite surface
5 first end
6 inner surface
7 bite surface protrusion
8 bite surface edge portion
9 lower portion of tongue trough
10 suction channel connector
11 suction tube
12 suction trough
13 recess
14 groove of suction trough
15 second end
100 tooth and tongue support
L lower bite surface level

1. A tooth and tongue support comprising
a tongue support element with a trough for the tongue, the trough having a concave inner surface and a convex outer surface; and
a tooth support element with an upper and a lower bite surface arranged on an outer surface side of the tongue trough; in which
the upper bite surface coincides with the lower bite surface, but on an opposite side of the tongue trough;
a first end of the inner surface of the tongue trough is positioned between the upper and the lower bite surfaces of the tooth support element; and
the inner surface of the tongue trough is directed sideways in relation to the tooth support element;
the inner surface of the tongue trough being thus arranged to receive the patient's tongue and to position it to a side of the oral cavity; and
the concave inner surface of the tongue trough being arranged to position the tongue partly between the bite surfaces.

2. A tooth and tongue support as claimed in claim 1, wherein the bite surface has a basic shape that is curved in a longitudinal direction thereof.

3. A tooth and tongue support as claimed in claim 1, wherein the bite surface comprises protrusions.

4. A tooth and tongue support as claimed in claim 3, wherein the protrusions are arranged in a transverse direction.

5. A tooth and tongue support as claimed in claim 1, wherein the bite surface comprises a raised edge portion (8).

6. A tooth and tongue support as claimed in claim 1, wherein the tongue trough has a lower portion that is arranged to extend past a level of the lower bite surface.

7. A tooth and tongue support as claimed in claim 1, comprising a suction channel connector arranged to the tongue support element and arranged to receive a suction tube.

8. A tooth and tongue support as claimed in claim 7, comprising a suction trough arranged into the tongue trough, the suction trough forming a recess that sets on the outer surface side of the tongue trough, to which recess said suction channel connector is connected and thus fluids collecting into the recess is removable through the suction tube.

9. A tooth and tongue support as claimed in claim 8, wherein the inner surface of the suction trough comprises at least one groove connecting with said recess.

10. A tooth and tongue support as claimed in claim 1, the support consisting of a single uniform piece of material.

11. A tooth and tongue support that comprises:
a tongue support element that has an outer surface that is at least partially convex;
an upper bite surface that is formed on an upper part of the outer surface of the tongue support element;
a lower bite surface that is formed on a lower part of the outer surface of the tongue support element, and is vertically aligned with and beneath the upper bite surface; and
a trough that is formed within the tongue support element, has a concave inner surface that is sized to receive the tongue in a position at least partially disposed between the upper and lower bite surfaces.

12. A tooth and tongue support that comprises:
a tongue support element that has an outer surface that is at least partially convex;
an upper bite surface that is formed on an upper part of the outer surface of the tongue support element;
a lower bite surface that is formed on a lower part of the outer surface of the tongue support element, and is vertically aligned with and beneath the upper bite surface;
a trough that is formed within the tongue support element, has a concave inner surface that is sized to receive the tongue in a position that is at least partially disposed between the upper and lower bite surfaces; and
a suction channel connector that is on the tongue support element and is receives a suction tube.
